(19) United States
(12) Patent Application Publication
(10) Pub. No.: US 2015/0320776 A1
(43) Pub. Date: Nov. 12, 2015

(54) METHOD OF INHIBITING HELICOBACTER PYLORI

(71) Applicant: CHUNG-YI LO, TAINAN CITY (TW)

(72) Inventor: CHUNG-YI LO, TAINAN CITY (TW)

(21) Appl. No.: 14/270,496

(22) Filed: May 6, 2014

(51) Int. Cl. A61K 31/69; A61K 31/045; A61K 31/194; A61K 31/19; A61K 31/192; A61K 31/196

(52) U.S. Cl. CPC A61K 31/69; A61K 31/192; A61K 31/196; A61K 31/194; A61K 31/19; A61K 31/045

(57) ABSTRACT

The present invention relates to a method of inhibiting Helicobacter pylori, principally using a compound A or a compound B for feedback inhibition of 3-dehydroquinate dehydratase in the shikimate pathway to inhibit Helicobacter pylori. The said compound A is one of the compounds as follows: benzoic acid, benzoic acid derivative, phenylacetic acid, phenylacetic acid derivative, trans-cinnaminic acid, 1,4-cyclohexanedicarboxylic acid, 4-indophenylboronic acid, phthalic acid and 3,4-dimethoxybenzyl alcohol. The said compound B is precursor of benzoic acid, benzoic acid derivative, phenylacetic acid or phenylacetic acid derivative generated by β-oxidation of fatty acids.

### BACKGROUND OF THE INVENTION

1. Field of the Invention

The present invention relates to a method of inhibiting Helicobacter pylori, especially to a method of using a compound similar to the chemical structure of 3-dehydroshikimate to inhibit Helicobacter pylori by a feedback inhibition of 3-dehydroquinate dehydratase in the shikimate pathway.

2. the Prior Arts

Helicobacter pylori is a Gram-negative spiral bacterium found in the epithelial cells of the stomach. It is the main pathogen that induces human gastritis. Many studies have proven that Helicobacter pylori is involved in a number of diseases, such as chronic gastritis, gastric ulcers and duodenal ulcers, even highly involved in cancers, such as gastric cancer and mucosa-associated lymphoid tissue lymphoma (MALToma). The World Health Organization classified the bacterium as group I carcinogen in 1994.

The concensus conference on the treatment of Helicobacter pylori suggests a triple rescue therapy in which the first-line medicines, a proton-pump inhibitor (omeprazole) and more than two antibiotics (clarithromycin, tetracycline, amoxicillin or metronidazole), are used, whereby resistance to amoxicillin and tetracycline has rarely been reported. However, due to the fact that tetracycline and amoxicillin are easily affected by the gastric conditions, such as pH value, i.e. highly acidic conditions are not suitable for the two antibiotics, as shown in Table 1 (Megraud F. & Lamouliatte H. Aliment. Pharmacol. Ther. 17:1333-1343(2003)), the minimal inhibitory concentration (MIC₉₀) increases respectively by four fold and eight fold at the ratio of pH 5.5 to 7.0 and this often leads to the situation in which their effects are difficult to be predicted when used in vivo. Clarithromycin is an important component in the triple therapy and its drug-resistance is not high. Likewise, it is also easily affected by the gastric conditions, such as pH value and hence the MIC₉₀ value increases by eight fold at the ratio of pH 5.5 to 7.0. The in vivo efficacy of metronidazole is the closest to its in vitro test results, whereby whether the drug is used can be decided according to drug susceptibility testing (Megraud and Lamouliatte, 2003). The drug resistance of metronidazole is usually the main reason for its treatment failure (Debets-Ossenkopp et al., 1999). It was reported recently that the prevalence rate of metronidazole in the western world was 10%-50% (Ling et al., 1996; Lopez-Brea et al., 1997; Megraud, 1997; Megraud and Doermann, 1998; van der Wouden et al., 1997). At present, the drug resistance ratios in the region of Eastern Taiwan are known as follows: metronidazole 51.9%, amoxicillin 36.1% and clarithromycin 13.5% (Hu et al., 2007). The way that solves problems of bacterial drug resistance is principally to change the antibiotic. For instance, moxifloxacin or levofloxacin is used to replace metronidazole that shows severe drug resistance (Schrauwen et al., 2009; Yoon et al., 2009), or an additional antibiotic is directly added to the therapy to form a quadruple rescue therapy (Cheng and Hu, 2009). The common side effects, including nausea, diarrhea, constipation, vomiting and abdominal discomfort, are predominantly associated with the use of antibiotics. This can be chiefly ascribed to alteration in intestinal bacterial flora.

TABLE 1
Minimal inhibitory concentration (MIC) of various antibiotics
against susceptible H. pylori according to pH value

| | MIC₉₀ (mg/l) | | |
|---|---|---|---|
| Agent | pH 7.5 | pH 6.0 | pH 5.5 |
| Penicillin | 0.03 | 0.5 | 0.5 |
| Ampicillin | 0.06 | 0.25 | 0.5 |
| Cefalexin | 2 | 16 | 32 |
| Erythromycin | 0.06 | 2 | 8 |
| Clarithromycin | 0.03 | 0.06 | 0.25 |
| Ciprofloxacin | 0.12 | 0.5 | 2 |
| Tetracycline | 0.12 | 0.25 | 0.5 |
| Nitrofurantoin | 1 | 2 | 2 |
| Metronidazole | 2 | 2 | 2 |
| Bismuth subcitrate | 16 | 8 | — |

### SUMMARY OF THE INVENTION

The present invention provides a method of inhibiting Helicobacter pylori by using a compound A for a feedback inhibition of 3-dehydroquinate dehydratase in the shikimate pathway, wherein the said compound A is one of the compounds as follows: benzoic acid, benzoic acid derivative, phenylacetic acid, phenylacetic acid derivative, trans-cinnaminic acid, 1,4-cyclohexanedi-carboxylic acid, 4-indophenylboronic acid, phthalic acid and 3,4-dimethoxybenzyl alcohol.

Wherein the said benzoic acid derivative is selected from the group that consists of 4-nitrobenzoic acid, 4-(dimethylamino)benzoic acid, 2,4-dinitrobenzoic acid, 4-ethylbenzoic acid, 3-amino-4-methylbenzoic acid and 4-methylthiobenzoic acid.

Wherein the said phenylacetic acid derivative is selected from the group that consists of 4-aminophenylacetic acid, 4-bromophenylacetic acid, 4-chlorophenylacetic acid and 2-methoxyphenylacetic acid.

Wherein the minimal bactericidal concentration of benzoic acid against H. pylori J99 is 0.5 mg/ml.

Wherein the minimal bactericidal concentration of phenylacetic acid against H. pylori J99 is 1 mg/ml.

Wherein benzoic acid, benzoic acid derivative, phenylacetic acid or phenylacetic acid derivative of the said compound A is generated from a compound B via β-oxidation of fatty acids and the said compound B is precursor of benzoic acid, benzoic acid derivative, phenylacetic acid or phenylacetic acid derivative.

The present invention has the advantage that the foregoing compound A or compound B is not easily affected by pH value in the stomach and hence capable of keeping a stable effect on bacterial inhibition.

### BRIEF DESCRIPTION OF THE DRAWINGS

FIG. 1: a two-dimensional electrophoretogram treated with PBS.

FIG. 2: a two-dimensional electrophoretogram treated with benzoic acid.

FIG. 3: a two-dimensional electrophoretogram treated with phenylacetic acid.

FIG. 4: a schematic diagram showing the conversion of 3-dehydroquinate to 3-dehydroshikimate in the shikimate pathway.

FIG. 5: a diagram showing the size of inhibition zones formed on the medium by treating respectively with 3,4-dimethoxybenzyl alcohol, 4-indophenylboronic acid, ethanol and H₂O.

FIG. 6: a diagram showing test results of various bactericidal concentrations of benzoic acid and phenylacetic acid directed to Helicobacter pylori.

### DETAILED DESCRIPTION OF THE PREFERRED EMBODIMENT

Taken the foregoing technical characteristics together, the main efficacy of the method of inhibiting Helicobacter pylori in the present invention can be explicitly demonstrated in the following embodiments.

The present invention provides a method of inhibiting Helicobacter pylori, using a compound A similar to the chemical structure of 3-dehydroshikimate for a feedback inhibition of 3-dehydroquinate dehydratase in the shikimate pathway, wherein the said compound A is one of the compounds as follows: benzoic acid, benzoic acid derivative, phenylacetic acid, phenylacetic acid derivative, trans-cinnaminic acid, 1,4-cyclohexanedicarboxylic acid, 4-indophenylboronic acid, phthalic acid and 3,4-dimethoxybenzyl alcohol, and wherein the said benzoic acid derivative is selected from the group that consists of 4-nitrobenzoic acid, 4-(dimethylamino)benzoic acid, 2,4-dinitrobenzoic acid, 4-ethylbenzoic acid, 3-amino-4-methylbenzoic acid and 4-methylthiobenzoic acid, and wherein the said phenylacetic acid derivative is selected from the group that consists of 4-aminophenylacetic acid, 4-bromophenylacetic acid, 4-chlorophenylacetic acid and 2-methoxyphenylacetic acid, and wherein the dosage form of the compound A may be oral tablet, oral solution, injection solution, capsule or liposome.

Furthermore, benzoic acid, benzoic acid derivative, phenylacetic acid or phenylacetic acid of the foregoing compound A can be generated from a compound B by β-oxidation of fatty acids and the compound B is precursor of benzoic acid, benzoic acid derivative, phenylacetic acid or phenylacetic acid derivative and the dosage form of the compound B may be oral tablet, oral solution, injection solution, capsule or liposome.

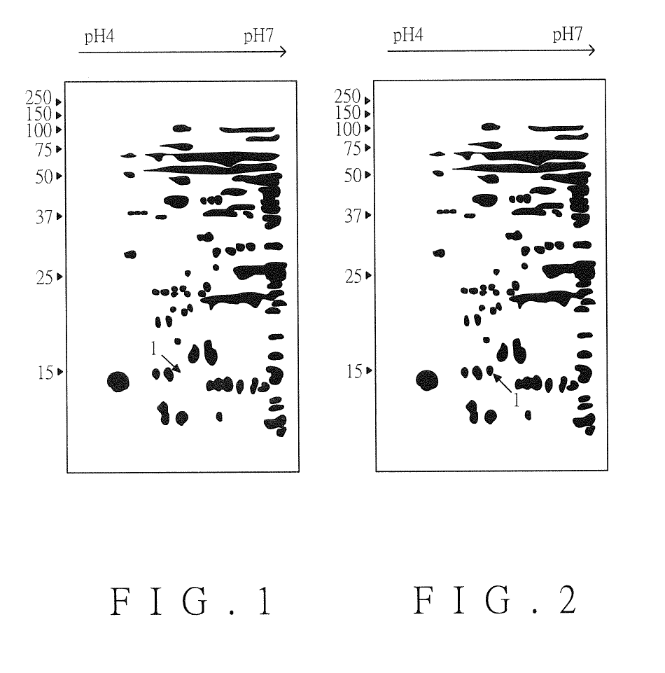

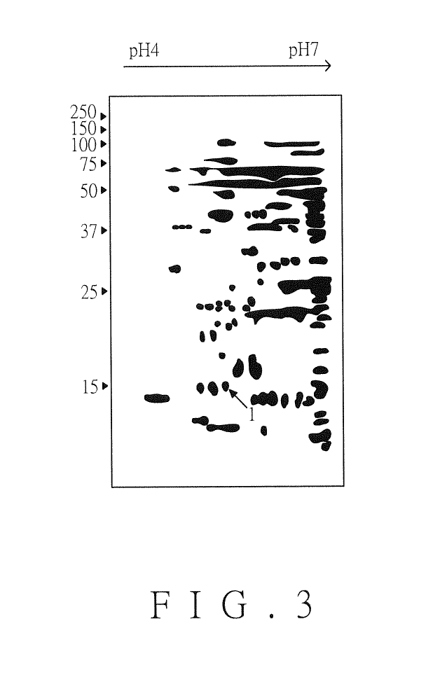

As shown in FIG. 1, FIG. 2 and FIG. 3, two-dimensional electrophoretograms treated respectively with PBS, benzoic acid and phenylacetic acid were demonstrated, wherein the x-axis represents the value of PH, and the Y axis represents the molecular weight (unit is kDa). From these three two-dimensional electrophoretograms treated respectively with PBS, benzoic acid and phenylacetic acid, protein spots showing significant change in expression amounts were identified. These protein spots with significant difference were submitted to mass spectrum analysis and subsequently matched with the protein database (NCBI database) to perform protein identification. Table 2 indicates the datarow of 3-dehydroquinate dehydratase (HP-aroQ). After having matched with results of mass spectrum analysis, the PI value and molecular weight of 3-dehydroquinate dehydratase in the protein database were in conformance to those of protein 1 in FIG. 1, FIG. 2 and FIG. 3. Table 3 indicates the sequence in conformance to protein land its related information (sequence coverage and matched peptide). Consequently, the protein 1 in FIG. 1, FIG. 2 and FIG. 3 is a protein of Helicobacter pylori, 3-dehydroquinate dehydratase (HP-aroQ), which shows an increase in expression.

TABLE 2
Representative proteins differentially expressed by H. pylori J99
in treated with PBs.

| | Accession | | | Mw |
|---|---|---|---|---|
| spot | number | Protein description | pI | (kDa) |
| 1 | YP_001909905 | 3-dehydroquinate dehydratase | 4.98 | 18483 |

TABLE 3
Match protein and cover peptide cover sequences.

| Match Protein | 3-dehydroquinate dehydratase |
|---|---|
| Peptide Sequence | MKILVIQGPNLNMLGHRDPRLYGMV |
| | TLDQIHEIMQTFVKQGNLDVELEFFQ |
| | TNFEGEIIDKIQESVGSDYEGIIINPGA |
| | FSHTSIAIADAIMLAGKPVIEVHLTNI |
| | QAREEFRKNSYTGAACGGVIMGFGP |
| | LGYNMALMAMVNILAEMKAFQEAQ |
| | QNNPNNPINNQK |
| Matched peptide | 3 |
| Sequence coverage | 10% |

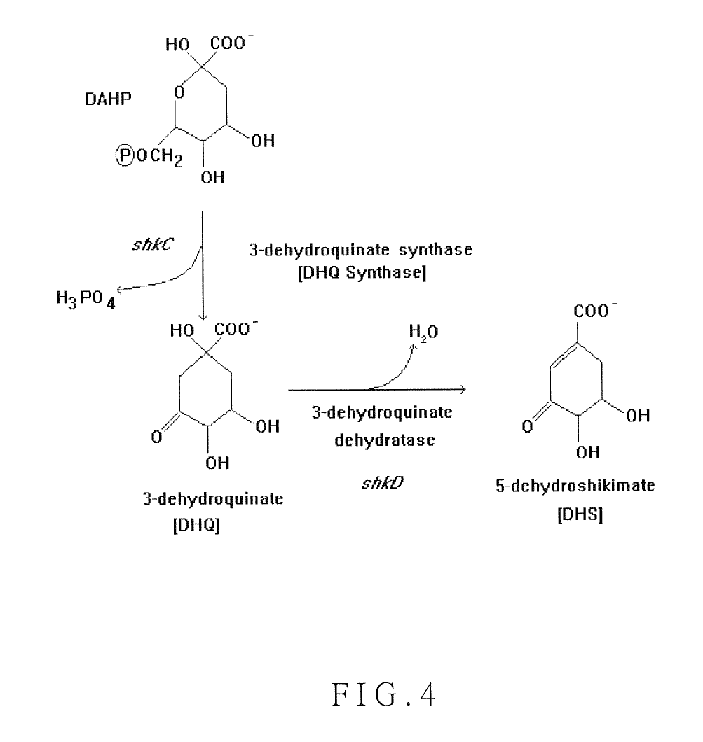

As indicated in FIG. 4, 3-dehydroquinate dehydratase is the third enzyme in the shikimate pathway, which converts 3-dehydroquinate to 3-dehydroshikimate. This pathway exists merely in bacteria, plants and several species of parasites and is inevitable for their survival. The main function of the pathway is to synthesize aromatic amino acids and aromatic compounds, including L-phenylalanine, L-tyrosine, L-tryptophan, folate cofactors, ubiquinone, vitamin E and vitamin K. However, there exists no such a pathway in mammals. In other words, the enzyme necessary for the pathway is missing in mammals. Therefore, the present invention is principally to use the foregoing compound A or compound B similar to chemical structure of 3-dehydroshikimate for feedback inhibition of 3-dehydroquinate dehydratase in the shikimate pathway to inhibit Helicobacter pylori. It should be particularly elucidated that the foregoing compound A or compound B reacts principally via benzene ring, hexacyclic ring or heterocyclic ring with active domain of 3-dehydroquinate dehydratase.

Helicobacter pylori J99 (HP J99) used in the embodiments of the present invention was purchased from Bioresource Collection and Research Center (BCRC). The cultivating condition is indicated as follows: the bacteria were inoculated onto CDC medium (CDC anaerobe 5% blood agar, BD) and then incubated at 37° C. for 48 to 72 hours. After incubation, the bacteria were further incubated under micro-aerobic condition (5% O₂, 10% CO₂, 85% N₂). For preparation of solutions with the foregoing compound A or compound B, ethanol was used as solvent to prepare various suitable concentrations.

The test on the inhibitory effect of Helicobacter pylori with the compound A or compound B of the present invention was carried out as follows: Helicobacter pylori was first grown on a CDC agar plate containing sheep blood for 48 to 72 hours. The bacterial colony was scraped with a cotton swab or an inoculation loop and subsequently transferred into a normal saline solution (0.9% NaCl). The optical density was then adjusted to 2 Mcfarland (1-4×10⁸/ml bacteria) to obtain a solution with Helicobacter pylori. Subsequently, the foregoing compound A or compound B was weighed out to prepare a series of concentrations (from 10 mg/ml to 100 mg/ml) of test solutions (40 μl) and then 40 μl of the solution were transferred onto blank paper discs. After dryness, the discs were separately stuck to a proper site on the agar plate. After incubation under micro-aerobic condition at 37° C. for 48 hours, observation was made if any inhibition zone emerged on the agar plate.

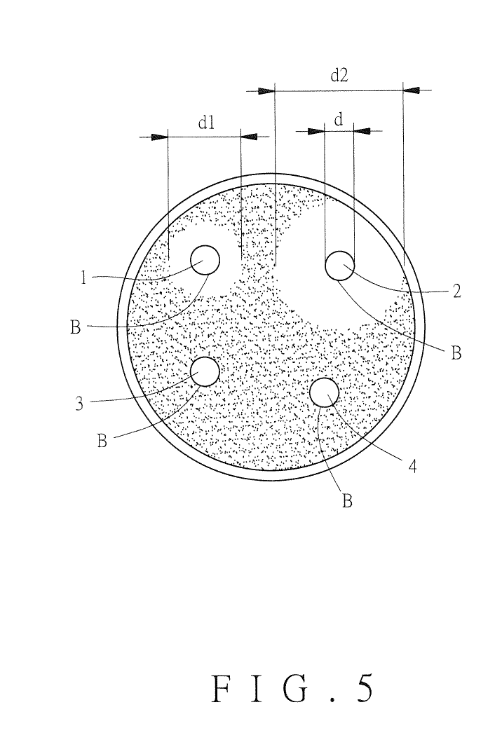

The compounds of the present invention, which were actually tested, included phenoxyacetic acid, 3,4-dihydrobenzoic acid, 3,5-diamino-benzoic acid, 4-aminophenylacetic acid, 4-nitrobenzoic acid, 2,4-dinitrobenzoic acid, 4-(dimethylamino)benzoic acid, 2-methoxyphenylacetic acid, 3-amino-4-methylbenzoic acid, 4-methylthiobenzoic acid, 4-bromphenylacetic acid, 1,4-cyclohexanedicarboxylic acid, phthalic acid, trans-cinnamic acid, 4-ethylbenzoic acid, phenylacetic acid, benzoic acid, 3-phenylpropionic acid, 4-phenylbutyric acid, 3,4-dimethoxybenzyl alcohol (1) and 4-indophenylboronic acid (2). Ethanol (3) and water (4) were used as control. FIG. 5 demonstrates the size of inhibition zones formed on the agar plate by treating with 3,4-dimethoxybenzyl alcohol (1), 4-indophenylboronic acid (2), ethanol (3) and water (4), wherein the diameter (d) of blank paper discs (BBL) was 8 mm. Since ethanol (3) and water (4) had no inhibitory effect, thus formation of inhibition zone around the blank paper discs (BBL) treated respectively with ethanol (3) and water (4) was not observed. However, two inhibition zones that were respectively (d1) 20 mm and (d2) 35 mm in diameter were formed around the blank paper discs (BBL) treated respectively with 3,4-dimethoxybenzyl alcohol (1) and 4-indophenylboronic acid (2). Table 4 indicates size of inhibition zone formed on the plate by treating the foregoing test compounds. Based upon the diameter of inhibition zone, it might be observed that 4-nitrobenzoic acid showed the strongest inhibitory ability against Helicobacter pylori. 1 mg of 4-nitrobenzoic acid dripped on a blank paper disc could form an inhibition zone with 70 mm in diameter. The other foregoing test compounds could also form different sizes of inhibition zone.

TABLE 4
Inhibition growth diameter obtained by diffusion method using
different concentrations of fatty acids to inhibit H. pylori (mm).

inhibition growth
diameter (mm)

| Agent | 1 mg/disc | 4 mg/disc |
|---|---|---|
| Phenoxyacetic acid | | 17 |
| 3,4-dihydrobenzoic acid | | 12 |
| 3,5-diaminobenzoic acid | | 12 |
| 4-aminophenylacetic acid | | 12 |
| 4-nitrobenzoic acid | 70 | |
| 4-bromphenylacetic acid | 16 | 35 |
| 4-(dimethylamino)benzoic acid | | 26 |
| 2,4-dinitrobenzoic acid | | 33 |
| 4-ethylbenzoic acid | 30 | |
| 3-amino-4-methylbenzoic acid | | 14 |
| 3-phenylpropionic acid | 14 | 34 |
| Trans-cinnamic acid | 25 | |
| 2-methoxyphenylacetic acid | | 16 |
| 4-methylthiobenzoic acid | 18 | 40 |
| phthalic acid | | 12 |
| 1,4-cyclohexanedicarboxylic acid | | 12 |
| Benzoic acid | | 24 |
| Phenylacetic acid | | 22 |
| 4-phenylbutyric acid | | 28 |
| 4-indophenylboronic acid | 35 | |
| 3,4-dimethoxybenzyl alcohol | | 20 |
| ethanol | | 8 |
| H₂O | | 8 |

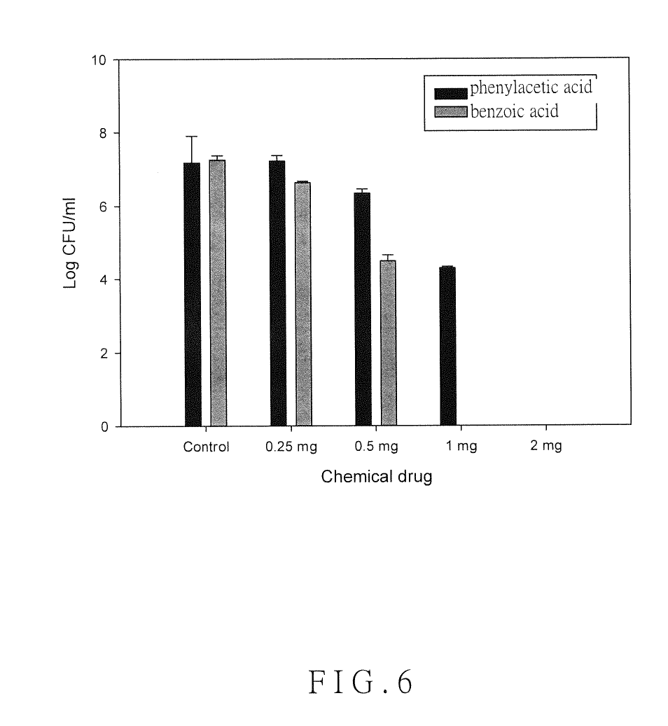

Minimal bactericidal concentration (MBC) was determined by incubating test bacteria with different doses of test compounds for a period of time. Subsequently, the solutions underwent serial dilutions and the bacteria were grown on the agar plate by plate coating. At this moment, the concentration of test compound lost its inhibitory ability because of being diluted via serial dilutions and plate coating and it could be seen from the clear bacterial solution if the bacteria were dead. If bacteria growth was inhibited by the concentration of test compound, the bacteria might continue to grow after the abovementioned treatment and appeared in the form of colonies. In an embodiment of the present invention, H. pylori J99 were pre-cultivated for two days and colonies were scraped and suspended in saline solution. The bacterial concentration was then adjusted to 2 McFarland. After addition of suitable concentrations of benzoic acid and phenylacetic acid and incubation at 37° C. for 1 to 2 hours, the bacterial suspension underwent serial dilutions. 100 μl aliquots were taken from each suspension and applied on CDC plates. The plates were incubated under micro-aerobic condition at 37° C. for two days. The number of bacterial colonies was noted down and the concentration of test compound, that is able to kill 99.9% of bacteria of control group, was used as minimal bactericidal concentration. Test results of minimal bactericidal concentrations of benzoic acid and phenylacetic acid against H. pylori J99 were demonstrated in FIG. 6, wherein the minimal bactericidal concentrations of benzoic acid and phenylacetic acid against H. pylori J99 were 0.5 mg/ml and 1 mg/ml respectively.

The foregoing compound A or compound B used in the present invention is not easily affected by pH value in the stomach and hence capable of keeping a stable effect on bacterial inhibition. As shown in Table 5, benzoic acid and phenylacetic acid were tested at pH 6.0 to 7.0. The result showed MIC values of benzoic acid and phenylacetic acid at pH 6.0 to pH 7.0 were 0.06 and 0.125 respectively and no change was observed. Therefore, these two compounds were substances that possessed a stable effect on bacterial inhibition under acidic conditions.

TABLE 5
Determination of minimum inhibition concentrations of benzoic
acid and phenylacetic acid against H. pylori J99 with different pH values

| | MIC₉₀ (mg/l) | | |
|---|---|---|---|
| Agent | pH 7.0 | pH 6.5 | pH 6.0 |
| Benzoric acid | 0.06 | 0.06 | 0.06 |
| Phenylacetic acid | 0.125 | 0.125 | 0.125 |

What is claimed is:

1. A method of inhibiting Helicobacter pylori, using a compound A for a feedback inhibition of 3-dehydroquinate dehydratase in the shikimate pathway, wherein the said compound A is one of the compounds as follows: benzoic acid, benzoic acid derivative, phenylacetic acid, phenylacetic acid derivative, trans-cinnaminic acid, 1,4-cyclohexanedi-carboxylic acid, 4-indophenylboronic acid, phthalic acid and 3,4-dimethoxybenzyl alcohol.

2. The method of inhibiting Helicobacter pylori according to claim 1, wherein the said benzoic acid derivative is selected from the group that consists of 4-nitrobenzoic acid, 4-(dimethylamino)benzoic acid, 2,4-dinitrobenzoic acid, 4-ethylbenzoic acid, 3-amino-4-methylbenzoic acid and 4-methylthiobenzoic acid.

3. The method of inhibiting Helicobacter pylori according to claim 1, wherein the said phenylacetic acid derivative is selected from the group that consists of 4-aminophenylacetic acid, 4-bromophenylacetic acid, 4-chlorophenylacetic acid and 2-methoxyphenylacetic acid.

4. The method of inhibiting Helicobacter pylori according to claim 1, wherein the minimal bactericidal concentration of the said benzoic acid against H. pylori J99 is 0.5 mg/ml.

5. The method of inhibiting Helicobacter pylori according to claim 1, wherein the minimal bactericidal concentration of the said phenylacetic acid against H. pylori J99 is 1 mg/ml.

6. The method of inhibiting Helicobacter pylori according to claim 1, wherein benzoic acid, benzoic acid derivative, phenylacetic acid or phenylacetic acid of the foregoing compound A can be generated from a compound B by β-oxidation of fatty acids and the compound B is precursor of benzoic acid, benzoic acid derivative, phenylacetic acid or phenylacetic acid derivative.
